(19) United States
(12) Patent Application Publication
(10) Pub. No.: US 2018/0193014 A1
(43) Pub. Date: Jul. 12, 2018

(54) EXPANDING SUTURE KNOT ANCHOR AND DEVICE FOR PLACEMENT OF SAME

(71) Applicant: Tack Surgical, LLC, Solana Beach, CA (US)

(72) Inventors: Charles P. Chalekson, Templeton, CA (US); Philip J. Simpson, Escondido, CA (US); David G. Matsuura, Del Mar, CA (US)

(21) Appl. No.: 15/864,915

(22) Filed: Jan. 8, 2018

Related U.S. Application Data
(60) Provisional application No. 62/443,474, filed on Jan. 6, 2017.

(51) Int. Cl. A61B 17/04

(52) U.S. Cl. CPC A61B 17/0401; A61B 17/0469; A61B 2017/0453; A61B 2017/0474; A61B 2017/0409

(57) ABSTRACT

The present invention includes a braided suture article adapted to be inserted through or into a tissue so as be able to exert a force on that tissue, the braided suture material adapted to be moved from an extended configuration to a collapsed configuration forming at least two loops extending substantially perpendicular to its threaded length and having a relatively rigid terminal end length that extends substantially perpendicular to the threaded length. The present invention further includes a suture placement device adapted to insert a length of suture material into or through a tissue to a space beyond a rear surface of said tissue so as to maintain said length of suture material, and a method of placing an expanding suture knot into a tissue.

### RELATED APPLICATION DATA

This application claims the priority benefit of U.S. Provisional Application No. 62/443,474, filed Jan. 6, 2017, and incorporates by reference the disclosures of U.S. Provisional Application No. 62/420,796, filed Nov. 11, 2016, and U.S. Provisional Application No. 62/453,304, filed Feb. 1, 2017, all of which are hereby incorporated in their entirety herein by reference.

### FIELD OF THE INVENTION

The present invention relates to tissue fixation and closure suture articles and suture placement devices for surgery and the like.

### BACKGROUND OF THE INVENTION

Surgical procedures can result in creation of a void around tissues that normally have direct contact and adherence with other tissues. The primary medical concern with respect to dead space is that fluid, or sometimes gas, can collect within this space. A seroma is the collection of this fluid within this potential space. Seromas most often occur at a surgical site where tissue has been intentionally elevated. Friction between these elevated layers, trauma to the tissue or an inflammatory response to foreign bodies such as implants or mesh may result in further production of this undesirable fluid.

Seromas can cause discomfort, damage surrounding tissue, compromise normal healing, create a substrate for infection, and cause troublesome aesthetic issues.

On an emotional level, seromas can be taxing for the patient and their care providers, causing anxiety and generating heavy demand for support in the form of office visits and phone calls.

Certain procedures are particularly prone to seroma formation, and result in extra precautions to prophylactically address that risk.

One option to decrease the risk is the placement of surgical drains, which may be left in place anywhere from a few days to multiple weeks. They require significant maintenance, are painful, and are mostly, but not completely effective at draining any fluid moving into the space. Drains do not prevent the production of fluid.

One option to close and remove dead space is the placement of internal quilting sutures at the time of surgery. This entails the closure of elevated tissue planes with a large number of meticulously placed sutures to close and obliterate dead space, preventing friction and establishing contact to minimize fluid production. In this spirit, post-surgical compression garments are applied to stabilize the tissue to limit edema and fluid production. In addition, the surgeon may frequently place one or more drainage tubes at the site.

Nonetheless, swelling and fluid can collect either immediately or in delayed fashion, even many weeks after surgery. This can result in additional comorbidity, medical cost and procedures for the patient, even jeopardizing the procedure success itself.

Procedures prone to seromas and for which drains are commonly used are numerous but are most frequently seen in both plastic surgery and general surgery. Surgeries that involve elevation and undermining of larger amounts of tissue tend to have higher risks toward seromas.

Some examples include breast surgeries such as lumpectomies, mastectomies, reductions, abdominoplasties, body lift procedures, hernia repairs, lymph node removal, tumor resections, and manipulation of major organs.

There are major costs and complications associated with post-surgical management of dead space and typically involve seromas, infection, and hematomas [bleeding]. Serious or long-term problems related to seromas are regarded as infrequent but can be costly, time-consuming, and require additional surgery and treatment.

However, there remains a need for efficient and effective tissue approximation in order to best reduce seroma formation, as well as to reduce or eliminate the need for draining. Specifically, there remains a need for effective and relatively inexpensive suture anchors for tissue fixation and approximation.

### SUMMARY OF THE INVENTION

In accordance with an aspect of the invention, the present invention includes suture articles, suture placement devices for tissue approximation and the like, and methods of tissue fixation and approximation.

### Suture Article—Expanding Knot

The suture article of the present invention may be described generally as including a suture article adapted to be inserted through or into a tissue so as to attach to the tissue, the suture article comprising: (a) a braided suture material comprising the following portions: (i) a principal suture material portion having a braided structure and an interferent terminal end length; (ii) a threaded length reverse-threaded through the braids of the principal suture material portion so as to form a braided suture arrangement comprising at least two loops; such that the braided suture material may be moved from an extended configuration wherein the intrerferent terminal end length, and the loops are substantially collateral with the principal suture material portion, and a collapsed configuration wherein the at least two loops and the terminal end length extend away from the principal suture material portion.

As used herein, formatives of the term “braid” or “braided” refers to the property of a suture material that permits the material to be effectively reverse threaded through itself so as to form the arrangements described herein. These may include traditional braided materials or other looped or apertured filamentous materials, including monofilament materials, that likewise may be reverse threaded through itself so as to form the arrangements described herein.

The suture article of the present invention may also be described as including a suture article adapted to be inserted through a tissue so as to be able to exert a force on the tissue, the suture article comprising: (a) a braided suture material comprising the following portions: (i) a relatively rigid terminal end length; (ii) a relatively flexible loopable length adapted to form at least two loops; and (iii) a threaded length reverse-threaded through the braids of the relatively flexible loopable length so as to create a distal cinch point between the relatively rigid terminal end length and the relatively flexible loopable length; such that the braided suture material may be moved from an extended configuration wherein the relatively rigid terminal end length, the relatively flexible loopable length and the threaded length are collateral or alongside one another (i.e., substantially parallel), and a collapsed configuration wherein the relatively flexible loopable length forms at least two loops extending from (i.e., substantially perpendicular to) the threaded length and the relatively rigid terminal end length extends from (i.e., substantially perpendicular to) the threaded length.

### Expanding Knot Suture Placement Device—Basic Device

The suture article placement device of the present invention may be described generally as including a suture placement device adapted to insert a length of suture material through a tissue to a space beyond a rear surface of the tissue so as to maintain the length of suture material within the tissue and resist removal thereof, the device comprising: (a) a containment tubular portion, the tubular portion slidingly containing: (b) a braided suture material having a distal end and comprising the following portions: (i) a principal suture material portion having a braided structure and an interferent terminal end length; (ii) a threaded length reverse-threaded through the braids of the principal suture material portion so as to form a braided suture arrangement comprising at least two loops; such that the braided suture material may be moved from an extended configuration wherein the interferent terminal end length, and the loops are substantially collateral with the principal suture material portion, and a collapsed configuration wherein the at least two loops and the terminal end length extend away from the principal suture material portion; and (c) a cinching pusher slidingly contained within the containment tubular portion and adapted to slidingly contain the threaded length and to urge the relatively rigid terminal end length toward the distal end of the braided suture material.

The suture placement device may additionally comprise a pusher adapted to urge the braided suture material to a position outside the containment tubular portion.

The suture placement device may additionally comprise an additional tubular portion slidably containing the containment tubular portion.

The suture article placement device of the present invention also may be described as including a suture placement device adapted to insert a length of suture material into the internal mass of or through a tissue to a space beyond a rear surface of the tissue so as to maintain the length of suture material within or against the tissue and resist removal thereof, the device comprising: (a) a containment tubular portion, the tubular portion slidingly containing: (b) a braided suture material comprising the following portions: (i) a relatively rigid terminal end length; (ii) a relatively flexible loopable length adapted to form at least two loops; and (iii) a threaded length reverse-threaded through the braids of the relatively flexible loopable length so as to create a distal cinch point between the relatively rigid terminal end length and the relatively flexible loopable length; such that the braided suture material may be moved from an extended configuration within the containment tubular portion wherein the relatively rigid terminal end length, the relatively flexible loopable length and the threaded length are maintained collateral or alongside one another (i.e., substantially parallel) by the containment tubular portion, and a collapsed configuration outside the containment tubular portion wherein the relatively flexible loopable length forms at least two loops extending from (i.e., substantially perpendicular to) the threaded length and the relatively rigid terminal end length extends from (i.e., substantially perpendicular to) the threaded length.

In one embodiment, the suture placement device additionally comprises a cinching pusher slidingly contained within the containment tubular portion and adapted to urge the braided suture material to a position outside the containment tubular portion, and further to urge the relatively rigid terminal end length toward the distal end of the braided suture material so as to form a collapsed configuration in the suture article wherein the relatively flexible loopable length forms at least two loops extending from (i.e., substantially perpendicular to) the threaded length and the relatively rigid terminal end length extends from (i.e., substantially perpendicular to) the threaded length. The cinching pusher may be of any shape so as to form these functions, but may be in the form of an additional tubular portion slidably containing the containment tubular portion.

### Method of Inserting an Expanding Knot Suture

The method of placing an expanding suture knot through a tissue to a space beyond a rear surface of the tissue so as to maintain the length of suture material within the tissue and resist removal thereof may be described generally as comprising: (a) obtaining access to a tissue having a facing surface and a rear surface, the rear surface adjacent to free space; (b) extending a braided suture material into the facing surface, through the rear surface and into the free space, the braided suture material comprising the following portions: (i) a principal suture material portion having a braided structure and an interferent terminal end length; (ii) a threaded length reverse-threaded through the braids of the principal suture material portion so as to form a braided suture arrangement comprising at least two loops; such that the braided suture material may be moved from an extended configuration wherein the interferent terminal end length, and the loops are substantially collateral with the principal suture material portion, and a collapsed configuration wherein the at least two loops and the terminal end length extend away from the principal suture material portion; and (c) collapsing the principal suture material portion into a collapsed configuration in the free space wherein the principal suture material portion forms at least two loops extending from the threaded length and the interferent terminal end length extends from the threaded length.

The method of placing an expanding suture knot through a tissue also may be described as including: (a) obtaining access to a tissue having a facing surface and a rear surface, the rear surface adjacent to free space; (b) extending a braided suture material into the facing surface, through the rear surface and into the free space, the braided suture material comprising the following portions: (i) a relatively rigid terminal end length; (ii) a relatively flexible loopable length adapted to form at least two loops; and (iii) a threaded length reverse-threaded through the braids of the relatively flexible loopable length so as to create a distal cinch point between the relatively rigid terminal end length and the relatively flexible loopable length; the braided suture material being in an extended configuration wherein the relatively rigid terminal end length, the relatively flexible loopable length and the threaded length are alongside one another (i.e., substantially parallel); and (c) collapsing the relatively rigid terminal end length and the relatively flexible loopable length into a collapsed configuration in the free space wherein the relatively flexible loopable length forms at least two loops extending from (i.e., substantially perpendicular to) the threaded length and the relatively rigid terminal end length extends from (i.e., substantially perpendicular to) the threaded length.

The collapse of the suture material may be brought about by drawing upon the threaded length or by urging the relatively rigid terminal end length so as to form the collapsed loop structure as described herein.

The present invention also includes a method of placing an expanding suture knot into a tissue so as to maintain the length of suture material within the tissue and resist removal thereof, that may be generally described as comprising: (a) obtaining access to a tissue having an internal mass; (b) extending a braided suture material into the internal mass, the braided suture material comprising the following portions: (i) a principal suture material portion having a braided structure and an interferent terminal end length; (ii) a threaded length reverse-threaded through the braids of the principal suture material portion so as to form a braided suture arrangement comprising at least two loops; such that the braided suture material may be moved from an extended configuration wherein the interferent terminal end length, and the loops are substantially collateral with the principal suture material portion, and a collapsed configuration wherein the at least two loops and the terminal end length extend away from the principal suture material portion; and (c) collapsing the principal suture material portion into a collapsed configuration in the free space wherein the principal suture material portion forms at least two loops extending from the threaded length and the interferent terminal end length extends from the threaded length.

The method of placing an expanding suture knot into a tissue so as to maintain the length of suture material within the tissue and resist removal thereof, also may be described as comprising: (a) obtaining access to a tissue having an internal mass; (b) extending a braided suture material into the internal mass, the braided suture material comprising the following portions: (i) a relatively rigid terminal end length; (ii) a relatively flexible loopable length adapted to form at least two loops; and (iii) a threaded length reverse-threaded through the braids of the relatively flexible loopable length so as to create a distal cinch point between the relatively rigid terminal end length and the relatively flexible loopable length; the braided suture material being in an extended configuration wherein the relatively rigid terminal end length, the relatively flexible loopable length and the threaded length are collateral or alongside one another (i.e., substantially parallel); and (c) drawing upon the threaded length so as to collapse the relatively rigid terminal end length and the relatively flexible loopable length into a collapsed configuration in the internal mass wherein the relatively flexible loopable length forms at least two loops extending from (i.e., substantially perpendicular to) the threaded length and the relatively rigid terminal end length extends from (i.e., substantially perpendicular to) the threaded length.

A partial list of materials frequently used in medical equipment and devices of this type (other than metals, many are available as USP Class VI) may include:

Metals:

300 Series Stainless Steel

Titanium

Nickel Titanium Alloys

Aluminum

Polymers:

Polycarbonate (PC)

Acrylonitrile butadiene styrene (ABS)

ABS/PC Copolymers

Acetyl (Delrin®, Celcon®)

Modified Acrylics

Polyether Ether Ketone (PEEK)

Polypropylene (PP)

Polyethylene (PE)

Poly Vinyl Chloride (PVC)

Polytetrafluoroethylene (PTFE)

Elastomers:

Thermoplastic Elastomers (TPE)

Thermoplastic Urethanes (TPU)

Fluoroelastomer (Viton®)

Silicone

Latex

Polyisoprene

Bio-Absorbable:

Polydioxanone (PDS)

Polyglycolic Acid PGA

Polylactic Acid (PLA)

Poly-L-lactic Acid (PLLA)

It will be appreciated that the optional handle, anchor-directing portions and the hollow cinching cannula/suture conduit may be produced from any material appropriate to the intended use whether sterile or non-sterile (i.e., for uses other than surgery or treatment, such as taxidermy or post-mortem use), and with due regard to disposability where desired. For instance, the anchor-directing portions and the hollow cinching cannula/suture conduit may be produced from metal, such as medical grade aluminum, while the handle portion may be produced from metal or plastics commonly used in medical devices, typically disposable ones.

The suture articles, suture placement devices and suture placement and tissue approximation methods of the present invention may be applied to procedures prone to seromas including various forms of plastic surgery, large tumor resections, and procedures involving repositioning of major organs. The most common types of surgery that result in seromas include breast procedures, abdominoplasty, body contouring and hernia repair. Other procedures where the present inventions may be advantageously applied include those where tissue approximation is required following the use of a trocar, such as procedures involving trocar placement through the abdomen during laparoscopic surgery. Still other procedures may be those involving tissue approximation associated with securing grafts and implants. In addition to the closure of dead space internally, there also exists a need for rapid and accurate closure of skin and dermis more superficially. The ability to prevent dehiscence of surgical incisions frequently relies upon meticulous repair of the dermal and subcuticular layer, which are the source of strength in surgical closure sites. Running dermal sutures are quicker in their application, but as failure can occur through either suture rupture or knot failure, the entire suture can ultimately fail as a result of an issue at a single point, resulting in dehiscence of the incision and operative site. Interrupted sutures are more accurate, stronger, and allow for independent control of tension at each individual suture location. This is however much more time consuming, which is amplified as the surgical incision length increases. There exists a need to be able to apply independent and accurate suture placement in rapid fashion within the dermis and/or subcuticular plane to facilitate quicker and more reliable wound closure.

The tissues that may be approximated through use of the subject suture articles, suture placement devices, and suture placement and tissue approximation methods may include tissues of any type having two or more generally opposed or adjacent portions that may be advantageously drawn together to a desired position.

It will be appreciated that the present invention may be applied to other fields for the tissue approximation, adjoining and fixture, such as in veterinary medicine, or providing embalming or taxidermy services, and the like.

### BRIEF DESCRIPTION OF THE FIGURES

While the specification concludes with claims particularly pointing out and distinctly claiming the present invention, it is believed that the present invention will be better understood from the following description in conjunction with the accompanying Figures, in which like reference numerals identify like elements, and wherein:

FIG. 1a is a perspective view of a suture article strand in an extended disposition in accordance with one embodiment of the present invention.

FIG. 1b is a perspective view of a suture article strand in a collapsed disposition in accordance with one embodiment of the present invention.

FIG. 2 is a photographic perspective view of a suture article strand in an extended disposition in accordance with one embodiment of the present invention.

FIG. 3 is a photographic perspective view of a suture article strand in a collapsed disposition in accordance with one embodiment of the present invention.

FIG. 4a is a perspective view of a suture article strand in a collapsed disposition in accordance with another embodiment of the present invention.

FIG. 4b is a schematic representation of the junction points along a suture article strand in a collapsed disposition in accordance with another embodiment of the present invention.

FIG. 5a is a perspective view of a suture article strand in an extended disposition in accordance with yet another embodiment of the present invention.

FIG. 5b is a perspective view of a suture article strand in a collapsed disposition in accordance with yet another embodiment of the present invention.

FIG. 6 is a photographic perspective view of a suture article strand in a collapsed disposition in accordance with yet another embodiment of the present invention.

FIG. 7 is a photographic perspective view of a suture article strand in a collapsed disposition in accordance with yet another embodiment of the present invention.

FIG. 8 is a photographic perspective view of a suture article strand in a collapsed disposition in accordance with yet another embodiment of the present invention.

FIG. 9a is a perspective view of a suture article strand in an extended disposition in accordance with yet another embodiment of the present invention.

FIG. 9b is a perspective view of a suture article strand in a collapsed disposition in accordance with yet another embodiment of the present invention.

FIG. 10a is a perspective view of a suture article strand in an extended disposition in accordance with yet another embodiment of the present invention.

FIG. 10b is a perspective view of a suture article strand in a collapsed disposition in accordance with yet another embodiment of the present invention.

FIG. 11a is a perspective view of a suture article placement device containing a suture article strand in an extended disposition, in accordance with yet another embodiment of the present invention.

FIG. 11b is a perspective view of a suture article placement device inserting a suture article strand and forming it into a collapsed disposition, in accordance with yet another embodiment of the present invention.

### DETAILED DESCRIPTION

In the following detailed description of the preferred embodiment, reference is made to the accompanying drawings that form a part hereof, and in which is shown by way of illustration, and not by way of limitation, a specific preferred embodiment in which the invention may be practiced. It is to be understood that other embodiments may be utilized and that changes may be made without departing from the spirit and scope of the present invention.

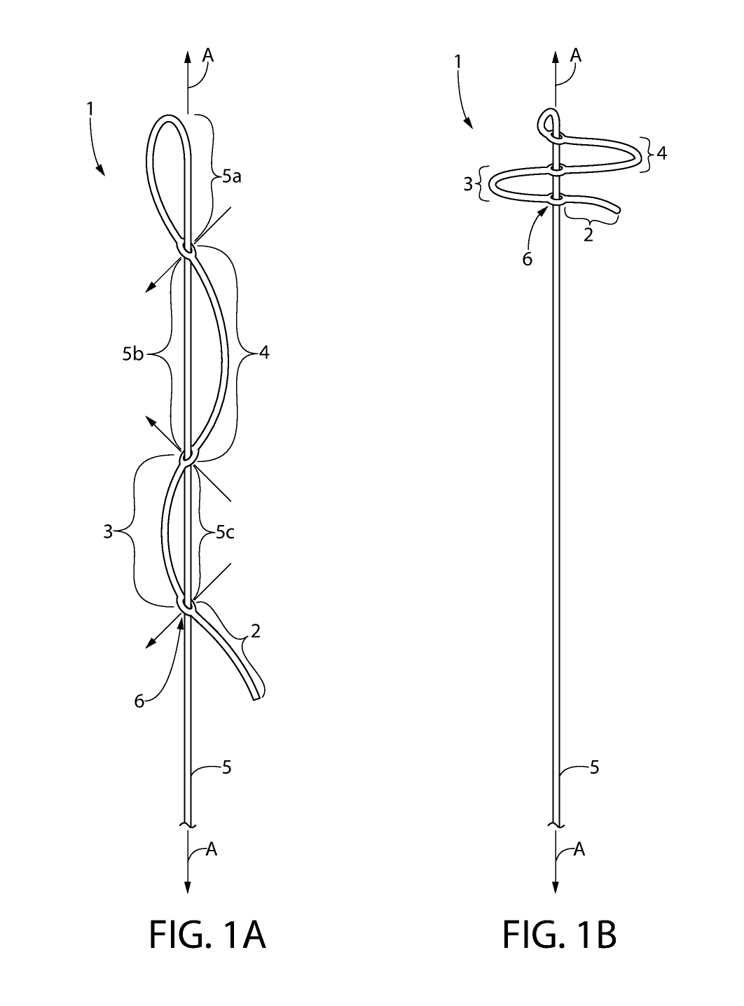

FIG. 1a is a perspective view of a suture article strand 1 in an extended disposition in accordance with one embodiment of the present invention. FIG. 1a shows suture article strand 1 made in this embodiment of a braided suture material comprising the following portions: (i) a relatively rigid terminal end length 2; (ii) a relatively flexible loopable length adapted to form at least two loops, such as loops 3 and 4; and (iii) a threaded length 5 (i.e., comprising 5a, 5b and 5c) reverse-threaded through the braids of the relatively flexible loopable length so as to create a distal cinch point 6 between the relatively rigid terminal end length 2 and the relatively flexible loopable length (i.e., forming loops 3 and 4). FIG. 1b is a perspective view of a suture article strand 1 in a collapsed disposition in accordance with one embodiment of the present invention. As may be appreciated from FIG. 1b, the braided suture material may be moved from an extended configuration in FIG. 1a wherein the relatively rigid terminal end length 2, the relatively flexible loopable length (i.e., forming loops 3 and 4) and the threaded length 5 are substantially collateral or alongside (i.e. substantially parallel), to a collapsed configuration wherein the relatively flexible loopable length forms at least two loops (i.e., loops 3 and 4) extending from (i.e., substantially perpendicular to) the threaded length 5 and the relatively rigid terminal end length 2 as well extends from (i.e., substantially perpendicular to) the threaded length 5. The orientation of the threaded portions of threaded length 5 (i.e., comprising 5a, 5b and 5c) out of the plane of the drawing, causes the two or more loops and the relatively rigid terminal end length 2 to orient in different planes, such that the collapsed configuration when viewed along the length of axis A of threaded length 5 resembles a multi-petal flower, wherein the flattened loops extend so as to resist movement of the cinched suture material from the tissue internal mass into which it has been inserted, or back through the surface through which it has been inserted into free space.

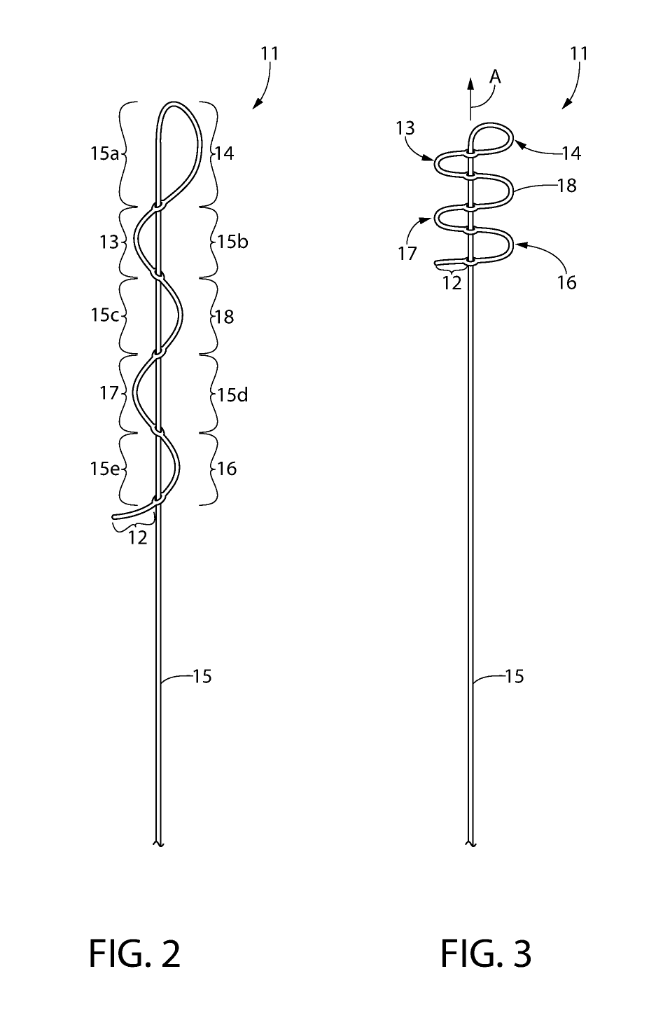

FIG. 2 is a photographic perspective view of a suture article strand in an extended disposition in accordance with one embodiment of the present invention, similar to the embodiment displayed in FIG. 1a. This view essentially shows the embodiment of FIG. 1a though with additional loops formed in the relatively flexible loopable length.

FIG. 2 shows suture article strand 11 made in this embodiment of a braided suture material comprising the following portions: (i) a relatively rigid terminal end length 12; (ii) a relatively flexible loopable length adapted to form at least two loops, such as loops 13, 14, 16, 17 and 18; and (iii) a threaded length 15 (i.e., comprising 15a, 15b, 15c, 15d, and 15e) reverse-threaded through the braids of the relatively flexible loopable length so as to create a distal cinch point 16 between the relatively rigid terminal end length 12 and the relatively flexible loopable length (i.e., forming loops 13, 14, 16, 17 and 18).

FIG. 3 is a perspective view of a suture article strand 11 in a collapsed disposition in accordance with one embodiment of the present invention. As may be appreciated from FIG. 3, the braided suture material may be moved from an extended configuration in FIG. 2 wherein the relatively rigid terminal end length 12, the relatively flexible loopable length (i.e., forming loops 13, 14, 16, 17 and 18) and the threaded length 15 are collateral or alongside (i.e. substantially parallel), to a collapsed configuration wherein the relatively flexible loopable length forms at least two loops (i.e., loops 13, 14, 16, 17 and 18) extending from (i.e., substantially perpendicular to) the threaded length 15 and the relatively rigid terminal end length 12 as well extends from (i.e., substantially perpendicular to) the threaded length 15. The orientation of the threaded portions of threaded length 15 (i.e., comprising 15a, 15b, 15c, 15d, and 15e) out of the plane of the drawing, causes the two or more loops and the relatively rigid terminal end length 12 to orient in different planes, such that the collapsed configuration when viewed along the length of axis A of threaded length 15 resembles a multi-petal flower, wherein the flattened loops extend so as to resist movement of the cinched suture material from the tissue internal mass into which it has been inserted, or back through the surface through which it has been inserted into free space.

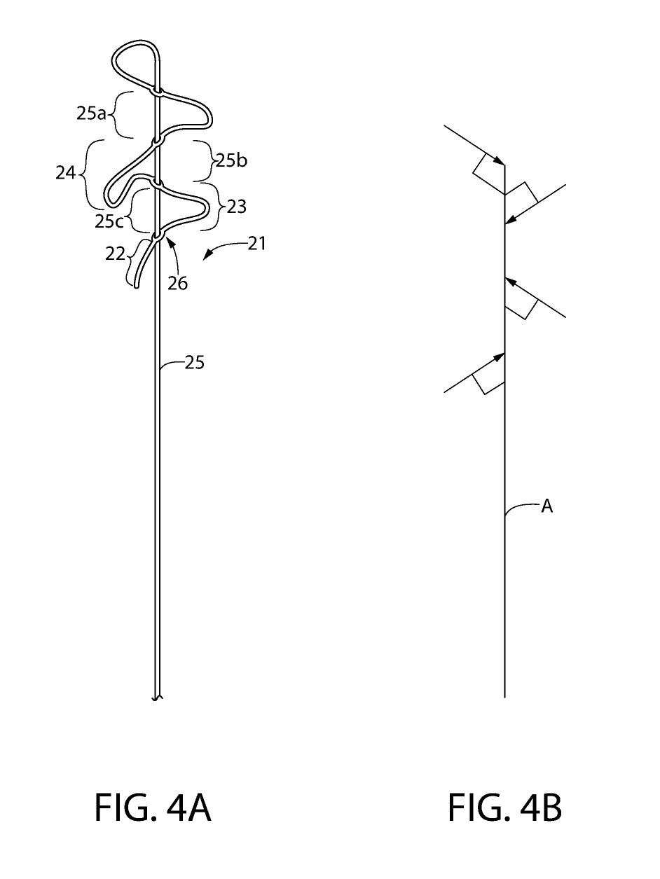

FIG. 4a is a perspective view of a suture article strand 21 in a collapsed disposition in accordance with another embodiment of the present invention. This embodiment shows a 90 degree clocked insertion. FIG. 4a shows suture article strand 21 made in this embodiment of a braided suture material comprising the following portions: (i) a relatively rigid terminal end length 22; (ii) a relatively flexible loopable length adapted to form at least two loops, such as loops 23 and 24; and (iii) a threaded length 25 (i.e., comprising 25a, 25b and 25c) reverse-threaded through the apertures of the relatively flexible loopable length so as to create a distal cinch point 26 between the relatively rigid terminal end length 22 and the relatively flexible loopable length (i.e., forming loops 23 and 24).

The braided suture material comprises the relatively rigid terminal end length 22, the relatively flexible loopable length (i.e., forming loops 23 and 24) and the threaded length 25 are shown having moved from an extended configuration wherein they are collateral or alongside one another (i.e., substantially parallel), to the displayed collapsed configuration wherein the relatively flexible loopable length forms at least two loops (i.e., loops 23 and 24) extending from (i.e., substantially perpendicular to) the threaded length 25 and the relatively rigid terminal end length 22 as well extends from (i.e., substantially perpendicular to) the threaded length 25.

The orientation of the threaded portions of threaded length 25 (i.e., comprising 25a, 25b and 25c) out of the plane of the drawing, causes the two or more loops and the relatively rigid terminal end length 22 to orient in different planes, such that the collapsed configuration when viewed along the length of axis A of threaded length 25 resembles a multi-petal flower, wherein the flattened loops extend so as to resist movement of the cinched suture material from the tissue internal mass into which it has been inserted, or back through the surface through which it has been inserted into free space.

FIG. 4b is a schematic representation of the junction points along a suture article strand and the respective angles of throughput with respect to axis A, in a collapsed disposition represented in FIG. 4a, in accordance with another embodiment of the present invention.

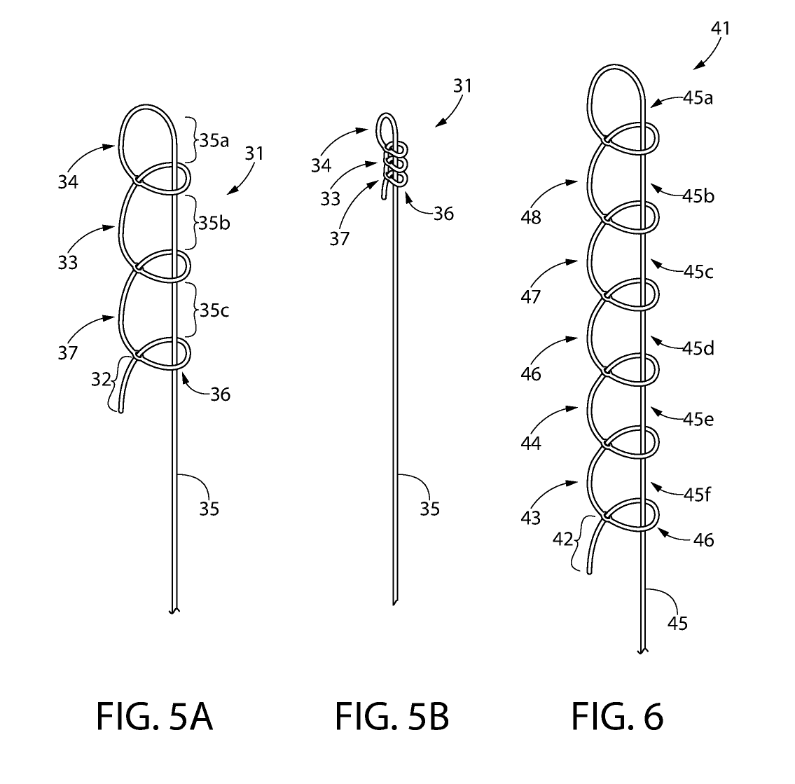

FIG. 5a is a perspective view of a suture article strand 31 in an extended disposition in accordance with yet another embodiment of the present invention. FIG. 5a shows suture article strand 31 made in this embodiment of a braided suture material comprising the following portions: (i) a relatively rigid terminal end length 22; (ii) a relatively flexible loopable length adapted to form at least two loops, such as loops 33, 34 and 37; and (iii) a threaded length 35 (i.e., comprising 35a, 35b and 35c) reverse-threaded through the apertures of the relatively flexible loopable length so as to create a distal cinch point 36 between the relatively rigid terminal end length 32 and the relatively flexible loopable length (i.e., forming loops 33, 34 and 37).

The orientation of the threaded portions of threaded length 35 (i.e., comprising 35a, 35b and 35c) out of the plane of the drawing, causes the two or more loops and the relatively rigid terminal end length 22 to orient in different planes, such that the collapsed configuration when viewed along the length of axis A of threaded length 25 resembles a multi-petal flower, wherein the flattened loops extend so as to resist movement of the cinched suture material from the tissue internal mass into which it has been inserted, or back through the surface through which it has been inserted into free space.

FIG. 5b is a perspective view of a suture article strand 31 in a collapsed disposition in accordance with yet another embodiment of the present invention wherein like reference numerals refer to the same portions thereof.

FIG. 6 is a photographic perspective view of a suture article strand in a collapsed disposition in accordance with yet another embodiment of the present invention, similar to the embodiment displayed in FIG. 5. This view essentially shows the embodiment of FIG. 5 though with additional loops formed in the relatively flexible loopable length.

FIG. 6 shows suture article strand 41 made in this embodiment of a braided suture material comprising the following portions: (i) a relatively rigid terminal end length 42; (ii) a relatively flexible loopable length adapted to form at least two loops, such as loops 43, 44, 46, 47 and 48; and (iii) a threaded length 45 (i.e., comprising 45a, 45b, 45c, 45d, 45e and 45f) reverse-threaded through the braids of the relatively flexible loopable length so as to create a distal cinch point 46 between the relatively rigid terminal end length 42 and the relatively flexible loopable length (i.e., forming loops 43, 44, 46, 47 and 48).

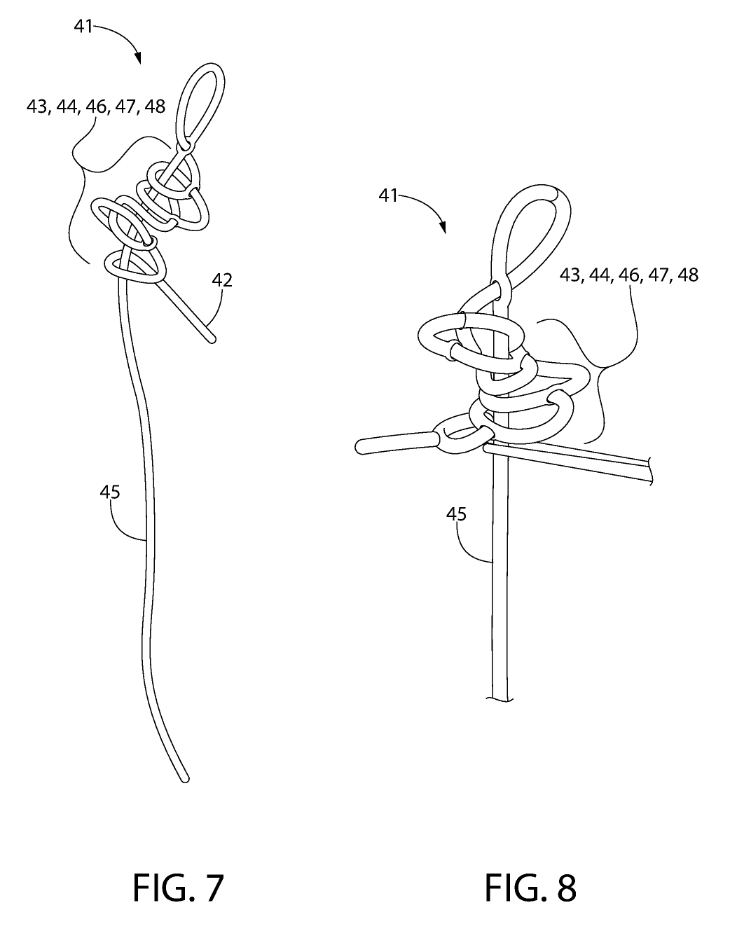

FIG. 7 is a detailed photographic perspective view of a suture article strand in a collapsed disposition in accordance with yet another embodiment of the present invention of FIGS. 5 and 6 wherein like reference numerals refer to the same portions thereof.

FIG. 8 is a further close-up detailed photographic perspective view of a suture article strand in a collapsed disposition as shown in FIG. 7 in accordance with yet another embodiment of the present invention of FIGS. 5 and 6 wherein like reference numerals refer to the same portions thereof.

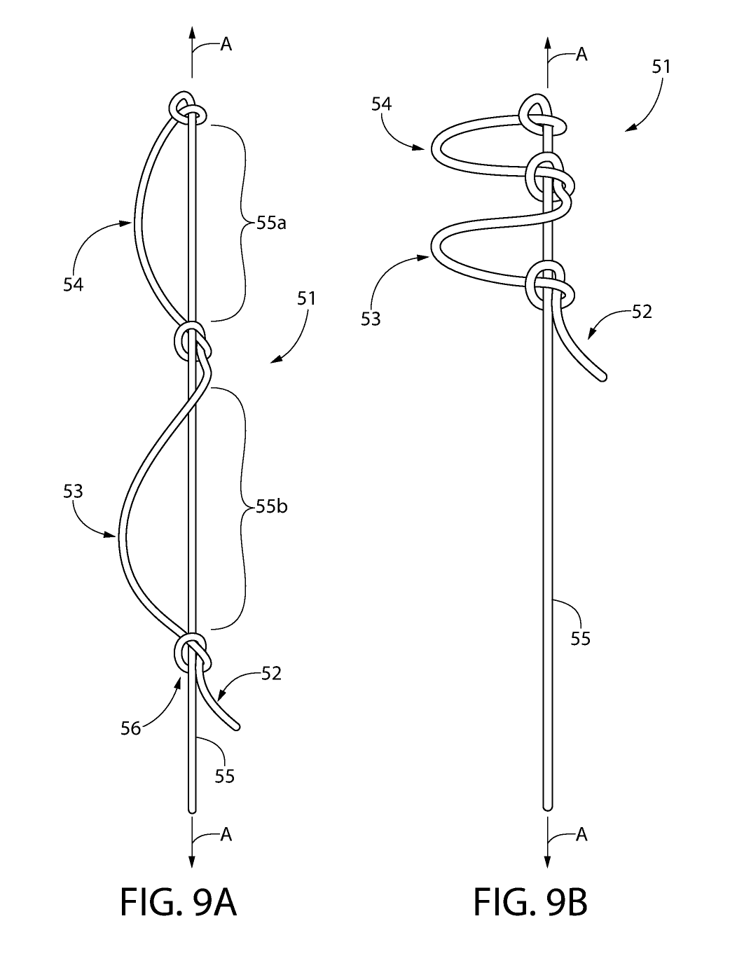

FIG. 9a is a perspective view of a suture article strand in an extended disposition in accordance with yet another embodiment of the present invention, showing suture article strand 51 in a collapsed disposition in accordance with yet another embodiment of the present invention. FIG. 9a shows suture article strand 51 made in this embodiment of a braided suture material comprising the following portions: (i) a relatively rigid terminal end length 52; (ii) a relatively flexible loopable length adapted to form at least two loops, such as loops 53 and 54; and (iii) a threaded length 55 (i.e., comprising 55a and 55b) reverse-threaded through the apertures of the relatively flexible loopable length so as to create a distal cinch point 56 between the relatively rigid terminal end length 52 and the relatively flexible loopable length (i.e., forming loops 53 and 54).

The orientation of the threaded portions of threaded length 55 (i.e., comprising 55a and 55b) out of the plane of the drawing, causes the two or more loops and the relatively rigid terminal end length 52 to orient in different planes, such that the collapsed configuration when viewed along the length of axis A of threaded length 55 resembles a multi-petal flower, wherein the flattened loops extend so as to resist movement of the cinched suture material from the tissue internal mass into which it has been inserted, or back through the surface through which it has been inserted into free space.

FIG. 9b is a perspective view of a suture article strand 51 in a collapsed disposition in accordance with yet another embodiment of the present invention wherein like reference numerals refer to the same portions thereof.

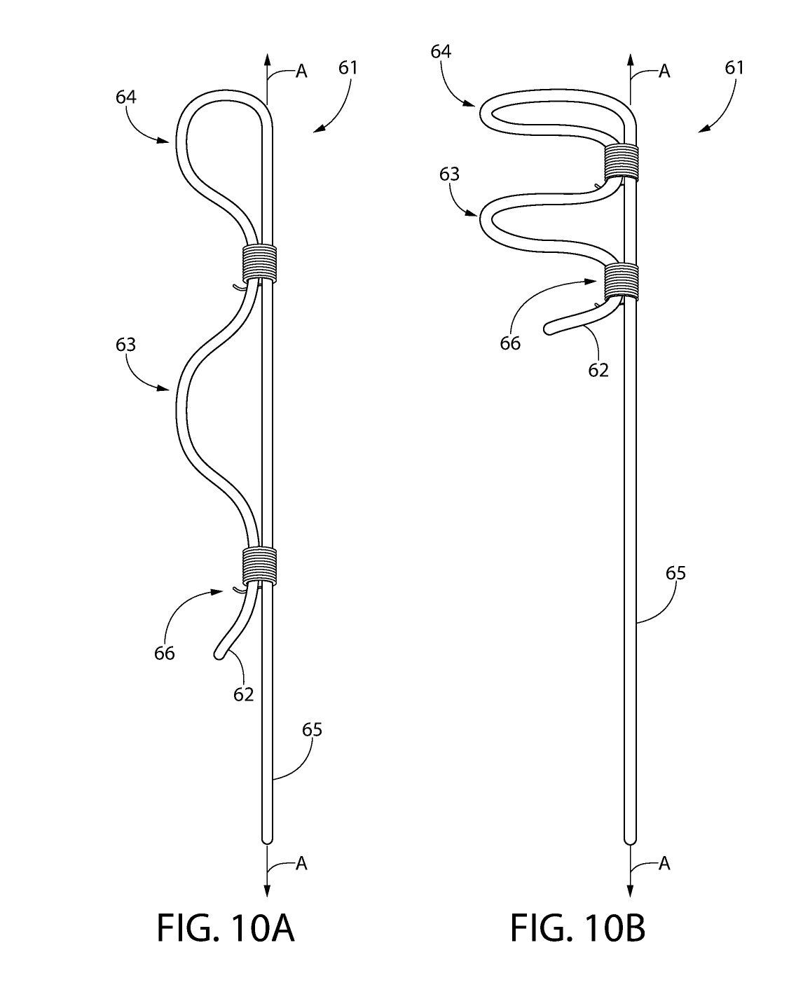

FIG. 10a is a perspective view of a suture article strand in an extended disposition in accordance with yet another embodiment of the present invention, showing suture article strand 61 in a collapsed disposition in accordance with yet another embodiment of the present invention. FIG. 10a shows suture article strand 61 made in this embodiment of a braided suture material comprising the following portions: (i) a relatively rigid terminal end length 62; (ii) a relatively flexible loopable length adapted to form at least two loops, such as loops 63 and 64; and (iii) a threaded length 65 reverse-threaded through the apertures of the relatively flexible loopable length so as to create a distal cinch point 66 between the relatively rigid terminal end length 62 and the relatively flexible loopable length (i.e., forming loops 63 and 64).

The orientation of the threaded portions of threaded length 65 out of the plane of the drawing, causes the two or more loops and the relatively rigid terminal end length 62 to orient in different planes, such that the collapsed configuration when viewed along the length of axis A of threaded length 65 resembles a multi-petal flower, wherein the flattened loops extend so as to resist movement of the cinched suture material from the tissue internal mass into which it has been inserted, or back through the surface through which it has been inserted into free space.

FIG. 10b is a perspective view of a suture article strand 61 in a collapsed disposition in accordance with yet another embodiment of the present invention wherein like reference numerals refer to the same portions thereof.

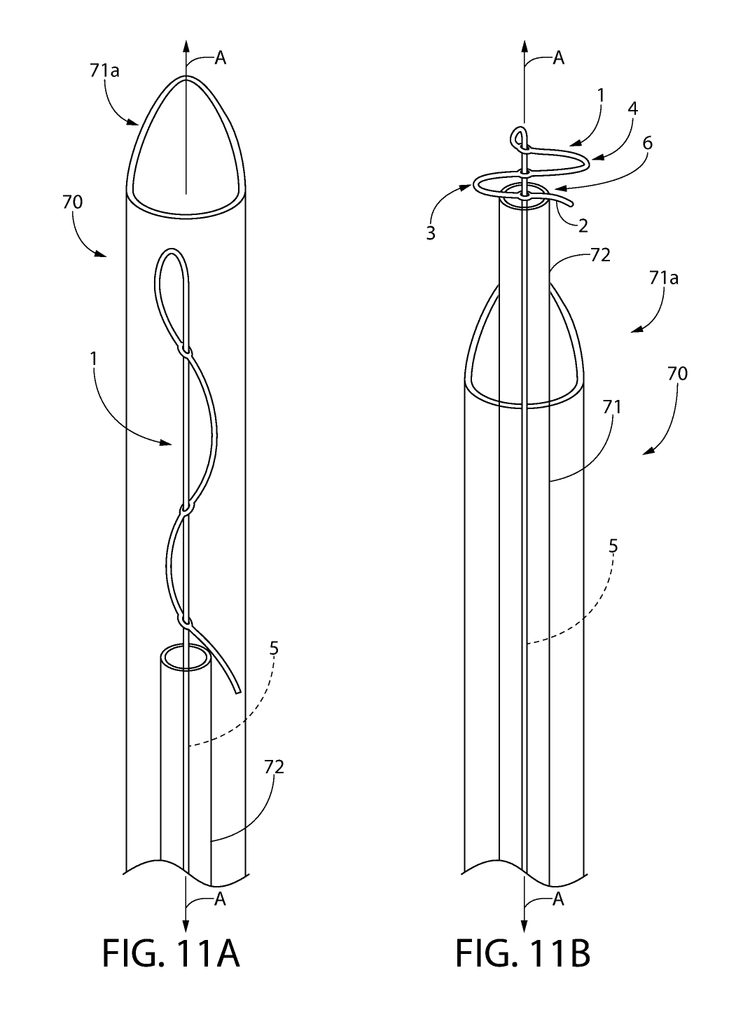

FIG. 11a is a perspective view of a suture article placement device containing a suture article strand in an extended disposition, in accordance with yet another embodiment of the present invention. FIG. 11a shows a suture placement device 70 adapted to insert a length of suture material (such as that shown and described in FIG. 1a) into a tissue or through a tissue to a space beyond a rear surface of the tissue, so as to maintain the length of suture material within or beyond the tissue, and resist removal thereof. The device comprises a containment tubular portion 71, the tubular portion slidingly containing a suture article strand 1 being in an extended configuration within the containment tubular portion 71, such that portions are maintained collateral or alongside one another (i.e., substantially parallel) by the containment tubular portion 71. The containment tubular portion 71 may be provided with a sharpened distal end 71a to better facilitate it being urged into or through tissue to be fixed.

The suture placement device 70 may be produced from any materials appropriate for the described surgical use, such as plastics and metals listed herein.

Once containment tubular portion 71 is placed into or through tissue to be fixed, the suture article strand 1 may be urged from within the containment tubular portion 71 along axis A by action of cinching pusher 72 by moving it distally, and ultimately to form the suture article strand 1 into the collapsed configuration outside the containment tubular portion 71, as shown and described in FIG. 1b.

FIG. 11b is a perspective view of a suture article placement device 70 inserting the distal end portion of a suture article strand 1 and forming it into a collapsed disposition. FIG. 11b shows the cinching pusher 72 engaging suture article strand 1 at relatively rigid terminal end length 2 so as to form the cinching point 6, ultimately urging the distal portion of suture article strand 1 from containment tubular portion 71, and whereby the relatively flexible loopable length forms at least two loops 3 and 4 extending from (i.e., substantially perpendicular to) the threaded length 5 and the relatively rigid terminal end length 2 extends from (i.e., substantially perpendicular to) the threaded length 5. The proximal end of threaded length 5 is secured by clamping or hand action so as to provide counterforce against the cinching action of the distal advance of the cinching pusher 72 to permit it to form the distal end portion of a suture article strand 1 into a collapsed disposition.

The sutures that may be produced in accordance with the present invention may have conventional monofilament or multifilament constructions. Examples of absorbable suture materials include absorbable polyester polymers and copolymers such as lactides, glycolides, polydioxanone, epsilon-caprolactone, polylactic acid, polyglycolic acid, and copolymers and blends thereof and equivalents thereof and the like, and may include conventional materials such as cat gut. Examples of nonabsorbable polymers include polyesters, silk, polyolefins such as polypropylene and polyethylene, nylon, and the like. Commercially available sutures that may be used with the stay suture devices of the present invention include the following sutures manufactured and sold by Ethicon, Inc., Somerville, N.J. 08876, USA such as Coated VICRYL Plus Suture, Coated VICRYL Suture, MONOCRYL Plus Suture, MONOCRYL Suture, PDS Plus Suture, PDS Suture, Surgical Gut Suture—Chromic, Surgical Gut Suture—Plain, PRONOVA Suture, ETHIBOND EXCEL Suture, ETHILON Suture, MERSILENE Suture, NUROLON Suture, PERMA-HAND Silk Suture, PROLENE Suture, and the like. The sutures will have a size that is effective to secure the tissue to be approximated in place, and the selection will be within the discretion of the surgeon. Although any size sutures may be used, typically the size of the sutures will range from a size of 2 to 6-0.

The foregoing is but one embodiment of the invention.

Other variations and embodiments of the devices and methods of the present invention include those that may be adapted for laparoscopic surgery or other types of minimally invasive surgery. Such device variants include those wherein the above-described handle portion may be eliminated in favor of using a simple hand-actuated hollow cinching cannula/suture conduit so as to reduce the width of the device to best accommodate laparoscopic surgery or keyhole surgery. In this embodiment, the hollow cinching cannula/suture conduit may be thumb actuated by the user, or may be fitted with a thumb wheel to allow the user to advance the hollow cinching cannula/suture conduit with respect to the anchor-directing portions, to cinch the suture lengths while counterforce is exerted and maintained, as otherwise described herein for manual operation involving use of the handle portion.

In similar fashion, other variations and embodiments of the invention include those devices and methods that may be adapted for robotic surgery. In this variant, the suture article placement device 70 may be affixed to a robotic arm that permits the containment tubular portion 71 and cinching pusher 72 to be moved as described above by moving the cinching pusher 72 distally, and ultimately forming the suture article strand 1 into the collapsed configuration outside the containment tubular portion 71, while counterforce is exerted and maintained upon the proximal portion of threaded length 5, as described herein for manual operation.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A. Suture Article—Expanding Knot
A suture article adapted to be inserted through or into a tissue so as to attach to said tissue, said suture article comprising:
a. a braided suture material comprising the following portions:
i. a principal suture material portion having a braided structure and an interferent terminal end length;
ii. a threaded length reverse-threaded through the braids of said principal suture material portion so as to form a braided suture arrangement comprising at least two loops;
such that said braided suture material may be moved from an extended configuration wherein said interferent terminal end length, and said loops are substantially collateral with said principal suture material portion, and a collapsed configuration wherein said at least two loops and said terminal end length extend away from said principal suture material portion.
B. Expanding Knot Suture Placement Device—Basic Device

2. A suture placement device adapted to insert a length of suture material through a tissue to a space beyond a rear surface of said tissue so as to maintain said length of suture material within said tissue and resist removal thereof, said device comprising:
a. a containment tubular portion, said tubular portion slidingly containing:
b. a braided suture material having a distal end and comprising the following portions:
i. a principal suture material portion having a braided structure and an interferent terminal end length;
ii. a threaded length reverse-threaded through the braids of said principal suture material portion so as to form a braided suture arrangement comprising at least two loops;
such that said braided suture material may be moved from an extended configuration wherein said interferent terminal end length, and said loops are substantially collateral with said principal suture material portion, and a collapsed configuration wherein said at least two loops and said terminal end length extend away from said principal suture material portion; and
c. a cinching pusher slidingly contained within said containment tubular portion and adapted to slidingly contain said threaded length and to urge said relatively rigid terminal end length toward the distal end of said braided suture material.

3. A suture placement device according to claim 1 additionally comprising a pusher adapted to urge said braided suture material to a position outside said containment tubular portion.

4. A suture placement device according to claim 1 additionally comprising an additional tubular portion slidably containing said containment tubular portion.
C. Method of Inserting an Expanding Knot Suture

5. A method of placing an expanding suture knot through a tissue to a space beyond a rear surface of said tissue so as to maintain said length of suture material within said tissue and resist removal thereof, said method comprising:
a. obtaining access to a tissue having a facing surface and a rear surface, the rear surface adjacent to free space;
b. extending a braided suture material into said facing surface, through said rear surface and into said free space, said braided suture material comprising the following portions:
i. a principal suture material portion having a braided structure and an interferent terminal end length;
ii. a threaded length reverse-threaded through the braids of said principal suture material portion so as to form a braided suture arrangement comprising at least two loops;
such that said braided suture material may be moved from an extended configuration wherein said interferent terminal end length, and said loops are substantially collateral with said principal suture material portion, and a collapsed configuration wherein said at least two loops and said terminal end length extend away from said principal suture material portion; and
c. collapsing said principal suture material portion into a collapsed configuration in said free space wherein said principal suture material portion forms at least two loops extending from said threaded length and said interferent terminal end length extends from said threaded length.

6. A method of placing an expanding suture knot into a tissue so as to maintain said length of suture material within said tissue and resist removal thereof, said method comprising:
a. obtaining access to a tissue having an internal mass;
b. extending a braided suture material into said internal mass, said braided suture material comprising the following portions:
i. principal suture material having a braided structure and an interferent terminal end length;
ii. a threaded length reverse-threaded through the braids of said principal suture material portion so as to form a braided suture arrangement comprising at least two loops;
such that said braided suture material may be moved from an extended configuration wherein said interferent terminal end length, and said loops are substantially collateral with said principal suture material portion, and a collapsed configuration wherein said at least two loops and said terminal end length extend away from said principal suture material portion; and
c. collapsing said principal suture material portion into a collapsed configuration in said tissue mass wherein said principal suture material portion forms at least two loops extending from said threaded length and said interferent terminal end length extends from said threaded length.
